(19) United States
(12) Patent Application Publication
(10) Pub. No.: US 2016/0318971 A1
(43) Pub. Date: Nov. 3, 2016

(54) NUCLEIC ACID COATED COLLOIDS

(71) Applicant: New York University, New York, NY (US)

(72) Inventors: David J. Pine, New York, NY (US); Marcus Weck, New York, NY (US); Yu Wang, New York, NY (US); Yufeng Wang, New York, NY (US); Xiaolong Zheng, New York, NY (US); Etienne Ducrot, New York, NY (US); Jeremy Yodh, New York, NY (US)

(73) Assignee: New York University, New York, NY (US)

(21) Appl. No.: 15/144,436

(22) Filed: May 2, 2016

Related U.S. Application Data
(60) Provisional application No. 62/155,185, filed on Apr. 30, 2015.

(51) Int. Cl. C07H 21/04

(52) U.S. Cl. CPC C07H 21/04

(57) ABSTRACT

DNA-coated colloids are introduced that can rearrange and anneal using single-stranded DNA coatings with thicknesses that are smaller than the colloidal particles, and with areal densities 5—than previously achieved. Micrometer-sized colloidal particles, such as poly(styrene), poly(methylmethacrylate) (PMMA), silica and titania, and 3-(trimethoxysilyl)propyl methacrylate (TPM), are coated with DNA by strain-promoted alkyne-azide cycloaddition. This enables growth of large colloidal crystals from a wide range of micrometer-sized DNA-coated colloids. When quenched from above to below the melting temperature, the rate of crystal formation exhibits the familiar maximum for intermediate temperature quenches observed in metallic alloys, but over a temperature range smaller by two orders of magnitude, owing to the highly temperature-sensitive diffusion between aggregated DNA-coated colloids.

### CROSS-REFERENCE TO RELATED PATENT APPLICATIONS

This application claims priority from Provisional Application U.S. Application 62/155,185, filed Apr. 30, 2015, incorporated herein by reference in its entirety.

### STATEMENT OF GOVERNMENT INTEREST

The United States Government has rights in the invention described herein pursuant to the MRSEC Program of the National Science Foundation under Award Number DMR-0820341. U.S. Army Research Office under MURI Grant Award no. W911NF-10-1-0518.

### SEQUENCE LISTING

The instant application contains a Sequence Listing which has been submitted electronically in ASCII format and is hereby incorporated by reference in its entirety. Said ASCII copy, created on Apr. 29, 2016, is named 046434-0520_SL.txt and is 2,337 bytes in size.

### FIELD OF THE INVENTION

The present invention generally relates self-assembling systems. In particular, the present invention relates to micrometer-sized colloidal particles coated with nucleic acids.

### BACKGROUND OF THE INVENTION

Colloidal particles coated with DNA hold great promise for the bottom-up self-assembly of heterogeneous structures for applications in plasmonics, magnetics, and photonics. By functionalizing particle surfaces with different combinations of complementary DNA, an almost limitless variety of colloidal structures can, in principle, be programmed. Realizing these structures depends on the ability of DNA-coated colloids not only to specifically bind but also to rearrange so that aggregated particles can anneal to achieve the structure that minimizes the free energy. Unfortunately, DNA-coated colloids generally collide and stick forming kinetically arrested random aggregates when the thickness of the DNA coating is much smaller than the particles.

Although the programmable self-assembly of DNA-coated nanoparticles (d<100 nm) have gained significant success, assembling larger micrometer-sized (d=200 nm˜4 um) DNA-coated colloids into three-dimensional crystals has proven much more difficult. The design principle and synthetic methodology for DNA-coated micrometer-sized particle is much less well developed in spite of optical applications that require larger particles and the obvious advantages of being able to study the crystallization kinetics by direct observation using an optical microscope.

The principle impediment has been that micrometer-sized DNA-coated colloids condense into random aggregates, but do not crystallize. In some cases, small crystallites form if the particles are smaller than a few hundred nanometers. More generally, however, when two or more DNA-coated particles bind, they have difficulty rolling over each other and become kinetically trapped. Thus, there is little if any relative diffusion between bound particles, which leads to the formation of random aggregates that are unable to anneal into crystals. A number of factors have been cited as contributing to the difficulty for bound DNA-coated colloids to diffuse so that they can anneal and form crystalline structures: the inhomogeneity of the interaction potential due to the random relatively sparse distribution of DNA strands on a colloid, the roughness of the colloid surface, and low areal density of DNA bound to the colloid surface.

### SUMMARY OF THE INVENTION

One embodiment of the invention relates to a method for fabricating coated colloidal particles comprising: synthesizing a plurality of micrometer size colloidal particles; and coating the plurality of colloidal particles with nucleic acids by applying strain-promoted alkyne-azide cycloaddition. The particle comprises dense and homogenous chlorine/azide surface functionalities.

Another embodiment relates to a colloidal crystal composition comprising: a colloidal particle having a plurality of strands of DNA attached thereto. Each strand of DNA comprises a 5′ end attached the colloidal particle, a 3′ terminus comprising a sticky end; and a flexible spacer extending therebetween comprising a plurality of base pairs. A plurality of colloidal particles are bound by binding of respectively associated DNA strands at the 3′ sticky end.

A further embodiment relates to a method to functionalize azide functionalized TPM/PS/PMMA/silica with DNA.

A further embodiment relates to a method to crystallize micrometer-sized colloidal particles driven by DNA interaction.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIGS. 1A-C show: (FIG. 1A) a general scheme of building colloidal crystal mediated by DNA hybridization; (FIG. 1B) a strain-promoted alkyne-azide cycloaddition reaction (SPAAC); and (FIG. 1C) DNA structures and sequence for sticky end. FIG. 1C discloses SEQ ID NOS 6, 1, 7, 1, and 8, respectively, in order of appearance.

FIGS. 2A-D show: (FIG. 2A) Synthetic scheme of Cl-functionalized PS particles; (FIG. 2B) SEM images of PS particles with smooth surfaces and different sizes; (FIG. 2C) 750 nm DNA-coated PS particles form bulk crystals of AB CsCl-like structure, the 100-plane is shown in the confocal image; and (FIG. 2D) 500 nm DNA-coated PMMA particles crystallize into FCC (left) or AB CsCl-type crystals, as revealed by confocal fluorescent image.

FIGS. 3A-E show: (FIG. 3A) Scheme showing the synthesis of Br-functionalized silica particle; (FIG. 3B) SEM image of silica particle indicates smooth particles surface; (FIG. 3C) FCC crystal structures formed by silica particles coated with DNA with palindrome sticky end; and AB CsCl colloidal crystals formed by silica particles coated with A and B complementary DNA; the 110-plane (FIG. 3D) and 100-plane (FIG. 3E) is shown in both bright-field and confocal fluorescent images.

FIGS. 4A-E show: (FIG. 4A) Scheme showing the synthesis of Cl-functionalized 3-(trimethoxysilyl)propyl methacrylate (TPM) particles, (FIG. 4B) FCC crystals of TPM particles showing the 111-plane and 100-plane, (FIG. 4C) FCC crystals show facets like molecular crystals, (FIG. 4D) AB CsCl-type crystals are shown by bright-field and confocal fluorescent images (FIG. 4E).

FIGS. 5A-F show: (FIG. 5A) SEM of commercial Cl-functionalized PS particles; (FIG. 5B) Bright-field and (FIG. 5C) confocal fluorescent images of rod-like structures formed by A- and B-functionalized PS particles with a low DNA coverage. SEM images showing silica particles with (FIG. 5D) smooth, (FIG. 5E) slightly rough, and (FIG. 5F)

FIGS. 6A-C shows: (FIG. 6A) AB CsCl-type binary colloidal crystals formed using PS and silica particles (left), or PS and TPM particles (right); (FIG. 6B) AlB₂ colloidal crystal can be fabricated using 1.0-um TPM and 500-nm PMMA particles; and (FIG. 6C) Cs₆C₆₀ crystals are formed when 1.5-um TPM and 500-nm PMMA particles with complementary DNA sticky end are employed.

FIGS. 7A-C show phase behavior of DNA-coated micrometer-sized particles. FIG. 7A is a schematic illustration of DNA-coated particles. The DNA is grafted to the particle surfaces using a strain-promoted alkyne-azide cycloaddition reaction (SPAAC). A 61-base long ‘polyT’ part, internally labeled with a Cy5 (red fluorescence) or Cy3 (green fluorescence) dye, serves as a flexible spacer. Particles were labeled with CGCG (palindrome) sticky ends and Cy5 dye as ‘P’, particles with GCAG as sticky ends and Cy3 dye as ‘A’, and particles with CTGC as sticky ends (complementary to A) and Cy5 dye as ‘B’. FIG. 7A discloses SEQ ID NOS 1 and 9, respectively, in order of appearance. FIG. 7B shows a singlet particle fraction (unbound particles) as a function of temperature. Particles are allowed to equilibrate at each temperature for 15 min prior to a measurement. The temperature window over which particle crystallization occurs is shaded. FIG. 7C is bright-field optical images showing the morphologies of 1.0-μm P particles: amorphous (left, 42° C.), crystalline (middle, 45° C.), and unbound (right, 48° C.). Scale bars, 5 μm.

FIGS. 8A-D shows colloidal crystals assembled from DNA-coated colloids. Confocal fluorescent images and corresponding drawings showing various crystals fabricated from DNA-coated colloids of different sizes and stoichiometries. FIG. 8A shows 1.0-μm P particles form face-centered cubic lattice, whose 111, 100, 110, and 311 planes are displayed. FIG. 8B shows an AB lattice (isostructural to CsCl) assembled from 1.0-μm A and 1.0-μm B particles with complementary DNA sticky ends. Multiple lattice planes are observed including, from left to right, 110, 100, 111, and 211 planes. FIG. 8C shows an AB₂ crystal (isostructural to AlB₂) is obtained using 1.0-μm A and 0.54-μm B particles. 100, 001, 111, and 101 planes are shown. FIG. 8D is an AB₆ crystal lattice (isostructural to Cs₆C₆₀) assembled from 1.5-μm A and 0.54-μm B particles. 110 and 100 planes are shown, accompanied by the corresponding red channel showing only the structural arrangement for the 0.54-μm particles. It is found that the AB₆ structure can tolerate a large number of B vacancies, up to 50%, at the surface of the crystals, while in the bulk there appear to be far fewer. Scale bars, 5 μm.

FIGS. 9A-H illustrate crystallization kinetics of DNA-coated colloids at various temperatures. FIGS. 9A-D are snapshots from a video showing the nucleation and crystal growth process of a CsCl lattice assembled from 0.54-μm A particles and 0.54-μm B particles at (FIG. 9A) 28.3° C., (FIG. 9B) 27.9° C., (FIG. 9C) 27.5° C., and (Figure D) 27.0° C. The melting temperature is 28.9° C. The time in minutes after quench is indicated at the upper left of each snapshot. FIG. 9E shows a mean square displacement [symbol] r² [symbol] of a particle rolling on a sphere at three different temperatures. The data are described by [symbol] r² [symbol] =At^{α} where 0.7<α≦1. FIG. 9F shows time-temperature-transformation diagram of the CsCl lattice showing the time required for 5%, 50% and 90% crystal conversion at four temperatures. The “C” shape indicates that the nucleation and overall transformation rate is maximized at intermediate temperatures and decreases when the temperature is either lowered or raised. The connecting lines are for illustration purpose only. FIG. 9G is a diagram showing that the crystal growth rate increases as temperature increases. The growth rate is measured as the average number of particle added to crystal per minute in the observation plane after nucleation. FIG. 9H shows the percentage of DNA-coated colloids converted to crystals after ten hours of annealing. The lateral reach Rₗ of the 61-base DNA is approximately 14 nm, as indicated by a dashed gray line. The mean distance d between grafted DNA sticky end is plotted as a function of DNA surface coverage, ranging from 5 nm to 22 nm. When the DNA coverage is below 10%, d exceeds Rₗ. In this limit, particles fail to completely crystallize. Scale bars, 5 μm.

FIGS. 10A-E show In-situ observations of crystal defect formation. Confocal fluorescent and schematic images showing in FIG. 10A, a grain boundary in the 110 plane of a Cs₆C₆₀ crystal and FIG. 10B, antisite as well as vacancy defects in a CsCl crystal. FIGS. 10C-E, Snapshots from videos showing the formation of crystal defects with FIG. 10C showing a three-particle vacancy defect is obtained at the intersection between two crystals and that a single particle vacancy defect forms as the crystal grows; FIG. 10D showing a green particle is trapped in a mismatched location when two crystals merge, leading to the formation of an anti-site defect; and FIG. 10E show grain boundaries form when two crystals merge while their orientations are not aligned. Scale bars, 5 μm.

FIGS. 11A-D illustrates TPM particles showing a scanning electron micrograph showing monodisperse colloids with (FIG. 11A), smooth surfaces, (FIG. 11B), rough surface. FIG. 11C is a Confocal fluorescent image showing colloids with a bright florescent corona, indicating a dense and uniform DNA coating. FIG. 11D, shows typical atomic force microscopy (AFM) scans of a smooth (left plots) and a rough particle (right plots). The particle roughness is extracted by calculating the root mean square (RMS) deviations from a perfectly smooth surface, shown by the blue lines. Scale bars, 1 μm.

FIGS. 12A-D shows colloidal crystals assembled from particles of various sizes. Bright-field microscope images showing FCC crystals assembled from (FIG. 12A) 0.54-μm, (FIG. 12B) 1.0-μm, (FIG. 12C) 2.0-μm, and (FIG. 12D) 3.5-μm particles coated with ssDNA bearing palindrome sticky ends. Scale bars, 10 μm.

FIGS. 13A-D illustrates colloidal crystals assembled from DNA coated colloids. Confocal fluorescent images showing a “full field of view” of various types of colloidal crystals. FIG. 13A shows a FCC lattice assembled from 1.0-μm P particles, verified by observations of several different crystalline planes. FIG. 13B shows a single crystal AB lattice (isostructural to CsCl) assembled from 1.0-μm particles with complementary DNA sticky ends. The sample is annealed at shallow quench for 50 hours. The green fluorescent particles are coated with ssDNA with A6 sticky ends (TGCGGT) and red fluorescent particles are coated ssDNA with B6 sticky ends (ACCGCA). FIG. 13C shows an AB₂ crystal (isostructural to AlB₂) is obtained using 1.0-μm A and 0.54-μm B particles. FIG. 13D, shows an AB₆ crystal lattice (isostructural to Cs₆C₆₀) assembles from 1.5-μm A and 0.54-μm B particles. Scale bars, 10 μm.

FIGS. 14A-C illustrates the mobility of bound DNA-coated colloids. FIG. 14A is a schematic illustration showing the process to affix a 2.0-μm A particle on a glass microscope slide. The DNA coated A particle is partially embedded in a thin polystyrene film spin-coated on the slide, with the assistance of THF/water vapor to plasticize the polymer film and let the particles sink. FIG. 14B is a scanning electron micrograph of the embedded particle. Roughly one hemisphere of the A particle is available for DNA binding. FIG. 14C, illustrates bright-field microscope images showing 1.0-μm B particles bounded via DNA hybridization to A particles. The blue lines represent the tracks B particles explore in six minutes on A particles at various quenching depth relative to the melting temperature; the surface area explored by the particle in a set period of time increases as the temperature increases. The mean square displacement [symbol] r² [symbol] is calculated from those tracks. Scale bars, 1 μm.

FIGS. 15A-D illustrates Temperature-dependent crystal sizes. Crystals assembled from 1.0-μm A and B particles at FIG. 15A, 26.0° C., FIG. 15B, 27.7° C., FIG. 15C, 28.6° C., and FIG. 15D, 29.3° C., illustrating the effect of annealing temperature on the size and conversion percentage of colloidal crystals. The sample is annealed for ten hours. Scale bars, 10 μm.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

Described herein and systems and methods that address the above mentioned problems by fabricating micrometer-sized particles with a high DNA grafting density, of single-stranded DNA, 5 to 25 times greater than previously reported, smooth surfaces, and short DNA sticky ends, with as few as four bases. These factors enable bound particles to roll over each other near the DNA melting temperature so that particles can find their free-energy minimum and, thus, form very large crystals. The ability to crystallize micrometer-size colloids allows one to follow the formation of crystals in real space and time. Different crystallization mechanisms have been observed, including nucleation and growth as well as spinodal decomposition, with kinetics spanning over a decade in time depending on the quench depth. In certain implementations, the crystallization kinetics are controlled by the sensitive temperature dependence of the surface diffusion of colloids bound by hybridized DNA. As further described below, the annealing process has been studied, including how various kinds of defects form.

Described in further detail below are synthetic schemes to fabricate DNA-coated micrometer-sized colloids and demonstrated that colloidal crystals can be produced possessing programmed compositions and structures. More complex structures, such as tertiary structures that have been achieved by DNA-coated nanoparticles, should be possible for micrometer-sized colloids by tuning the particles sizes and interactions. The particle synthesis and coating approach described herein should, in principle, be applied to integrate other functionalities on micrometer-sized particle surface. Examples include small molecules, polymers and bio-macromolecules such as protein.

It should be appreciated that the systems and methods herein can employ colloids made from a variety of materials, such as poly(styrene) (PS) and poly(methylmethacrylate) (PMMA), inorganic materials such as silica and titania, and hybrid materials such as 3-(trimethoxysilyl)propyl methacrylate (TPM) that can all be functionalized with nucleic acids. For example, colloids of each material can be synthesized with smooth surfaces having a dense layer of surface functional anchors for DNA attachment. A strain-promoted alkyne-azide cycloaddition reaction (SPAAC) is employed to functionalize the colloid surface with single-stranded DNA (ssDNA) with high efficiency. Colloids of the same or different kind, size, and DNA sequence can be mixed, allowing for the self-assembly of three-dimensional bulk colloidal crystals of a variety of structures and materials, including ones with either single or multiple materials of different sizes.

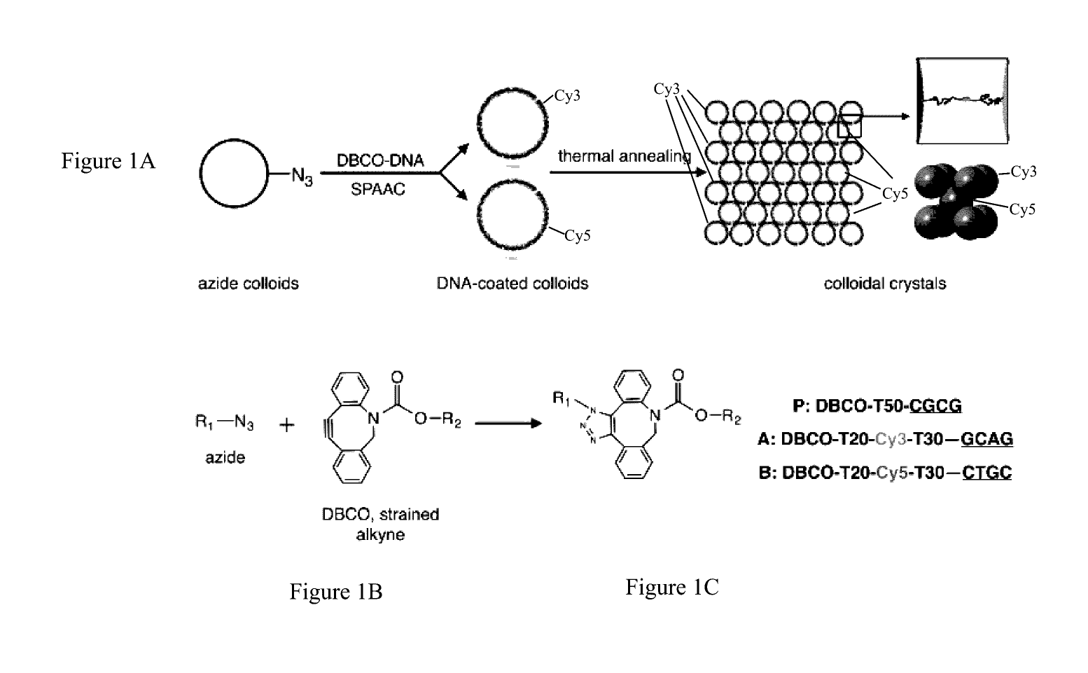

FIG. 1 illustrates the general scheme for assembling colloidal crystals through DNA hybridization. First, particles of the desired sizes are synthesized with a surface layer of halide groups, either chlorine or bromine, which can be converted quantitatively to azide groups for DNA grafting (FIG. 1A). To achieve a smooth particle surface and at the same time introduce a sufficient number of surface functional handles, different design and fabrication conditions are used depending on the material from which the colloid is made. A two-stage swelling and co-polymerization method was used to fabricate chlorine-functionalized PS and PMMA particles. Bromine functionalized silica particles are prepared via Stöber sol-gel process followed by 3-(bromopropyl)trichlorosilane (BTS) treatment. Chlorine functionalized organo-silica hybrid colloids are prepared from 3-(trimethoxysilyl)propyl methacrylate (TPM) through a combination of sol-gel process and emulsion polymerization. Strain-promoted alkyne-azide cycloaddition (SPAAC), sometimes referred to as ‘copper-free click’ chemistry, is employed to coat nucleic acids (in the examples provided herein, DNA) on the colloid surface.

SPAAC couples azides with a strained alkyne such as dibenzyl cyclooctyne (DBCO) forming a covalent triazole linkage (FIG. 1B). High conversion can be achieved in aqueous medium within a short period of time without addition of external catalyst. It has been used to conjugate biological molecules and macromolecules to surfaces, colloids, and with each other. Besides high efficiency and the stable nature of the covalent linkage, another advantage of SPAAC is its compatibility with high ionic strength and non-ionic surfactants. When grafting negatively charged DNA strands to colloidal surfaces, a certain amount of salt needs to be introduced to screen the DNA charges, thus reducing electrostatic repulsion between DNA strands and facilitating a high packing density of DNA on the surface. In one embodiment, 100 mM sodium chloride is utilized. To prevent aggregation of the colloids, a non-ionic surfactant may be utilized. For example, in one embodiment Triton X-100 with a trace amount of Pluronic F127 is used when conducting the functionalization using SPAAC. Under these conditions, in excess of 10⁵ DNA strands are grafted onto 1.0-μm particles, as measured by flow cytometry. Far fewer strands are detected when no salt is added.

In one embodiment, the nucleic acids are DNA, specifically ssDNA, each of which consists of three parts. At the 5′ end, attached using a DBCO group as a reactive handle for the SPAAC reaction with the azide functionalized particles. A 61-base long ‘polyT’ part, internally labeled with a fluorescent dye, serves as a flexible spacer. The 3′ terminus contains the sticky end to provide specific binding to complementary strands via DNA hybridization.

A collection of DNA-functionalized particles with different sizes and sticky ends are prepared. Particles functionalized with 4-base GCAG sticky ends and Cy3 dye (green) are called ‘A’, particles with 4-base CTGC sticky ends that are complementary to A and Cy5 dye (red) are called ‘B’, and particles with 4-base CGCG sticky ends that are self-complementary (palindrome) and Cy5 dye are called ‘P’ (FIG. 1A).

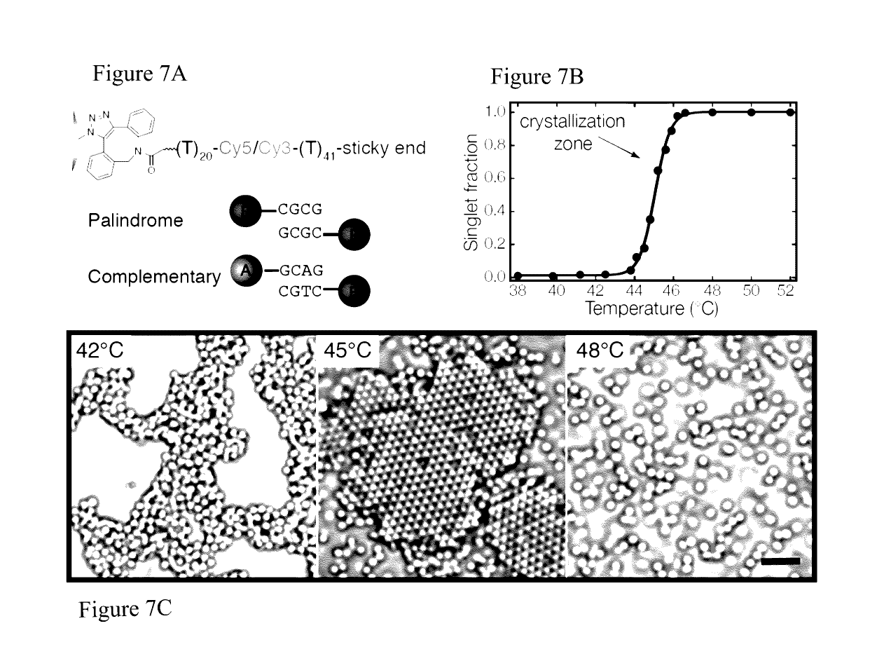

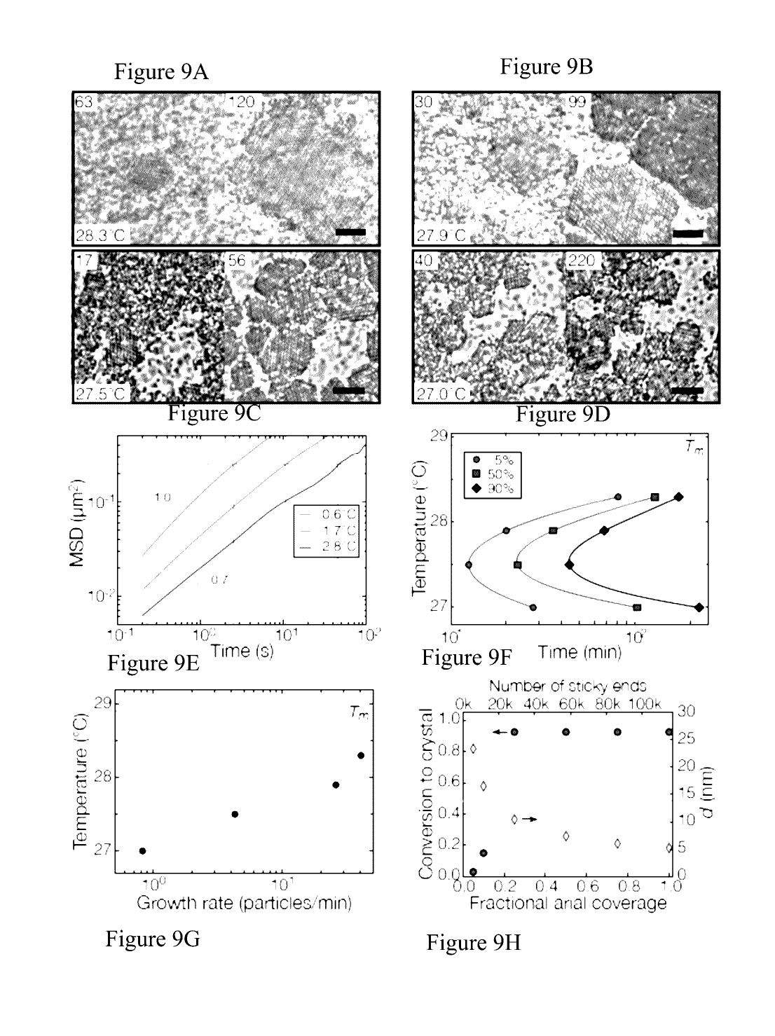

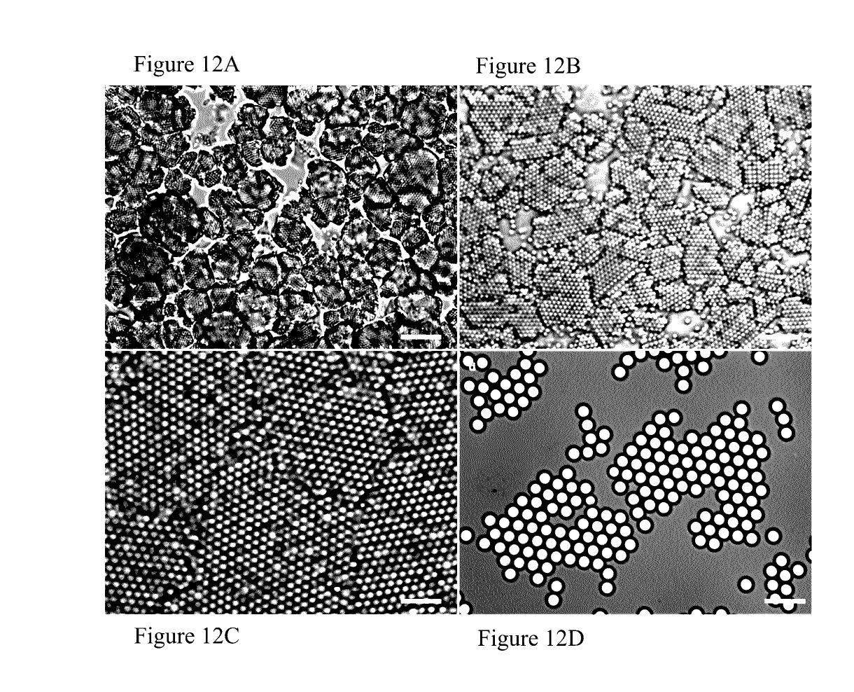

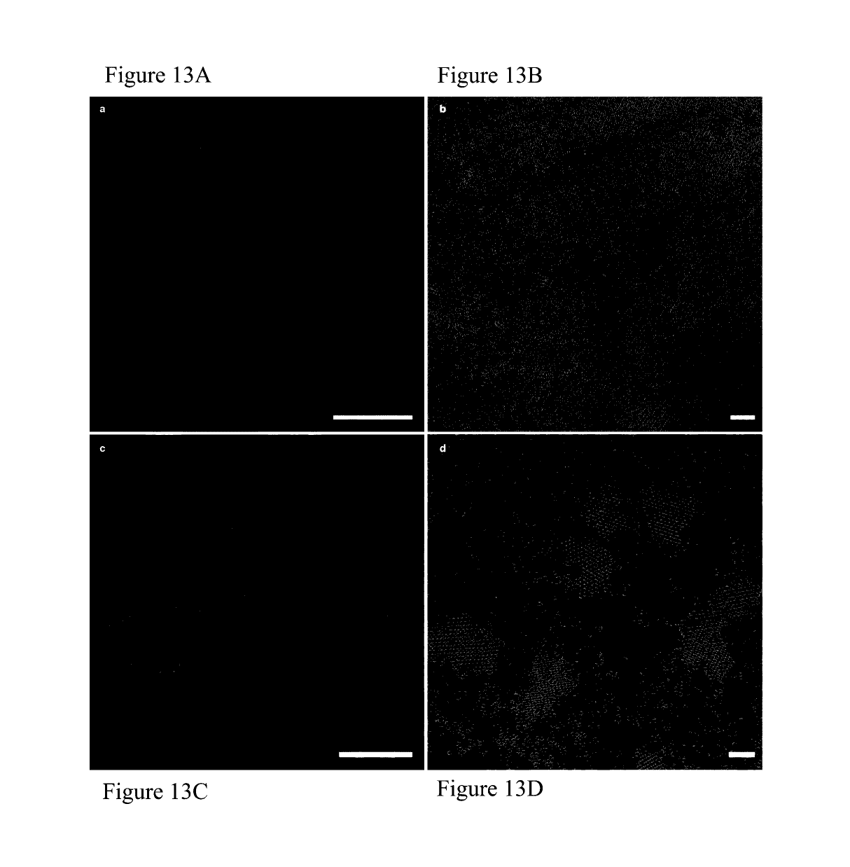

Particles coated with complementary DNA sticky ends reversibly associate and disassociate upon cooling below and heating above a certain melting temperature Tₘ (FIGS. 1b-c). For 1.0-μm P particles Tₘ=46.5° C. When quenched from above Tₘ to T=45° C., crystals spontaneously nucleate after about five minutes and grow to encompass almost the entire sample within 60 minutes (FIG. 7C, middle panel). Similar crystallization behavior is observed for all particle diameters investigated: 3.5 μm, 2.0 μm, 1.5 μm, 1.0 μm, and 0.54 μm (FIG. 9A, see also FIGS. 12, 13A). The crystals extend into three dimensions with the thickness depending primarily on the initial concentration of particles and the thermal gravitational height. In the present experiments the DNA is much shorter than the particle diameter, particles coated with double-stranded DNA never crystallize, whereas particles coated with single-stranded DNA always crystallize for shallow quenches.

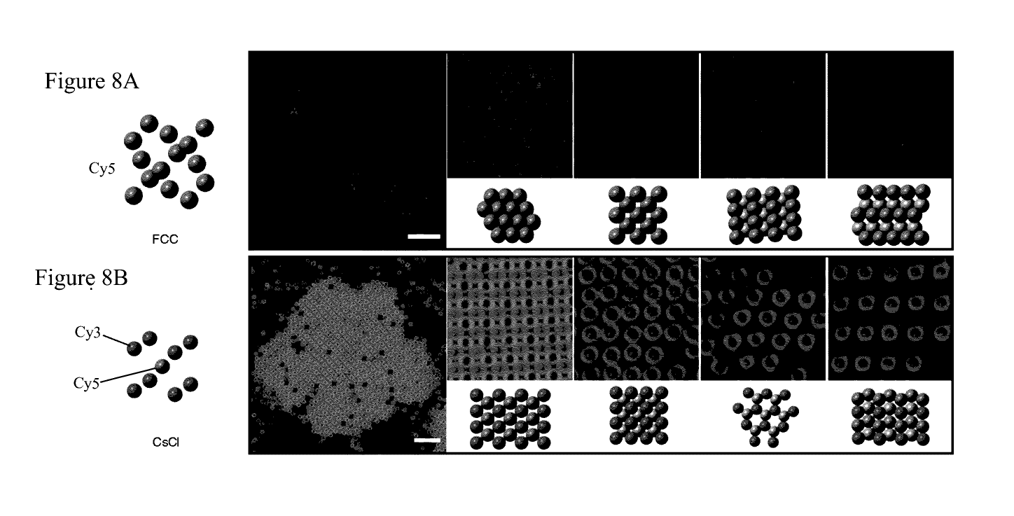

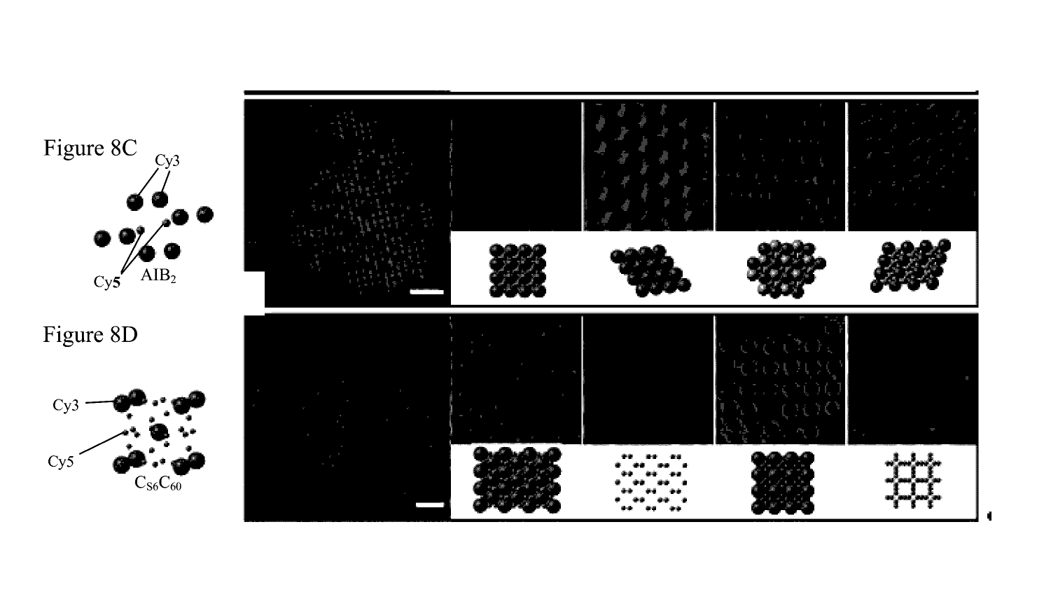

Binary systems were investigated that formed from A and B colloids bearing complementary sticky ends. For A and B particles of equal size, AB colloidal crystals isostructural to cesium chloride (CsCl) are obtained (FIG. 8B, FIG. 13B). Confocal fluorescent images of different crystal planes verify the structures. Images of the growth of AB crystals are shown in FIGS. 9A-D, and are discussed later.

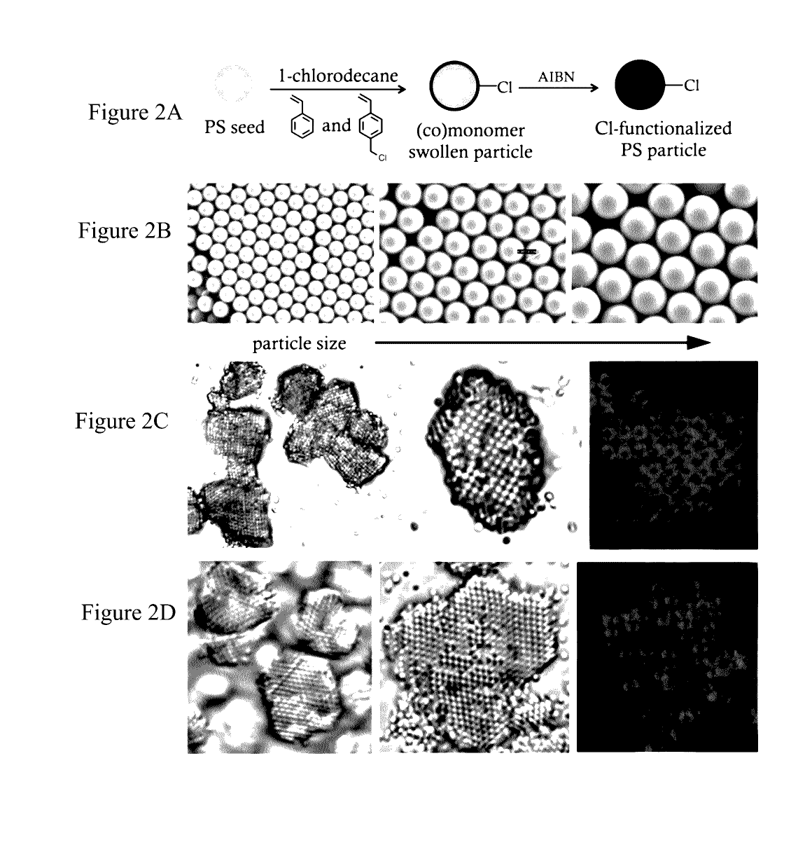

By changing the size ratio and stoichiometry of the A and B particles, AB₂ colloidal crystals are obtained isostructural to aluminum boride (AlB₂) (FIG. 8C, FIG. 13C) or AB₆ crystals isostructural to cesium fullerene-complex Cs₆C₆₀ (FIG. 9D, FIG. 13D). FIG. 2C shows the 100, 001, 111 and 101 planes of the AB₂ crystal formed using 1.0-μm A and 0.54-μm B particles. The images in FIG. 8D show the 110 plane and the 100 plane of an AB₆ crystal formed using 1.5-μm A and 0.54-μm B particles. These structures have also been observed for double-stranded DNA-coated gold nanoparticles, but where the DNA strand lengths are comparable to the nanoparticle diameters.

### Colloidal Particle Examples

Specific implementations were explored for colloidal particles according to the teachings above.

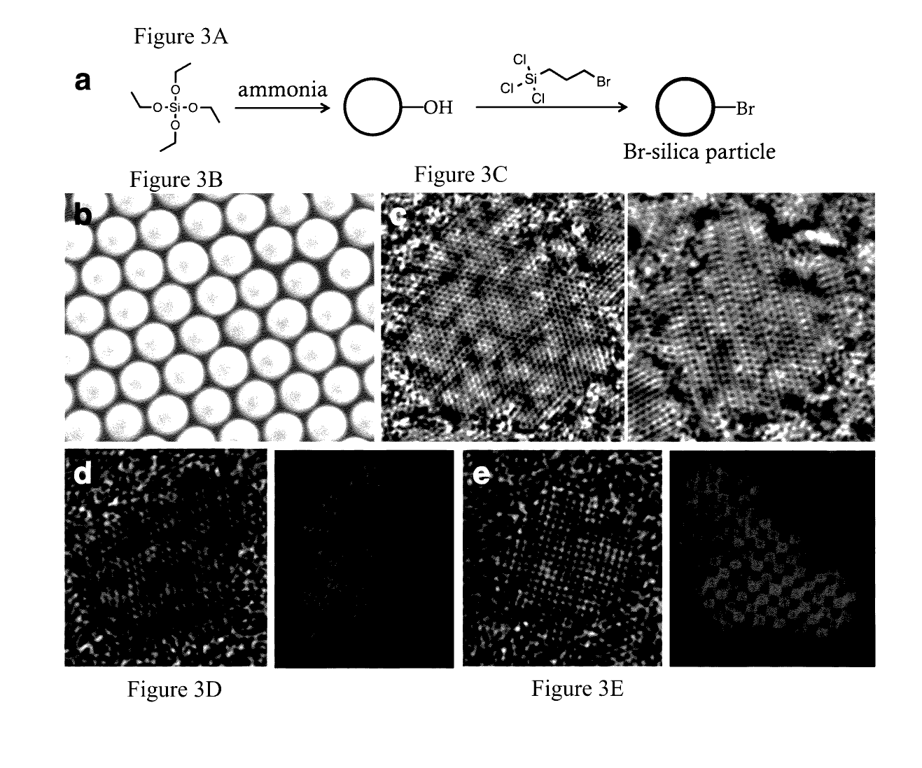

Polystyrene particles. In one embodiment, polystryrene particles are utilized as the colloidal particle. A two-stage swelling and co-polymerization method was used to fabricate chlorine-functionalized polystyrene particles (FIG. 2A). First, sulfonated PS seed particles, synthesized by emulsifier-free polymerization using styrene as monomer and potassium persulfate as initiator, are dispersed in aqueous solution containing sodium dodecyl sulfate (SDS, 0.3% w/w). 1-chlorodecane is then introduced as an oil-in-water emulsion pre-swelling the seed particles for 10 hours, followed by the addition of styrene along with a co-monomer, 4-vinylbenzyl chloride. The amount of 4-vinylbenzyl chloride is kept at a ratio of 10% to styrene, which insures the incorporation of enough chlorine groups on the particle surfaces while limiting possible phase separation between the two monomers when they are polymerized. An oil soluble initiator α,α′-Azobis(2-methylpropionitrile) (AIBN), is introduced at the same time. After another 10 hours, the temperature is raised to 78° C. to initiate the polymerization by thermally degrading AIBN, producing chlorine-functionalized PS particles. Particles of different sizes can be synthesized by consecutive swelling and growth in the same manner and using particles previously produced as seeds. All particles obtained had smooth surfaces, as shown from the scanning electron microscope (SEM) images in FIG. 3A for particle with sizes ranging from 500 nm to 1.3 μm. The chlorine groups on the particle surfaces are converted to azides by treating the particles with sodium azide at 70° C. overnight. Finally, single-stranded DNA (ssDNA) are attached using SPAAC, keeping the system at 55° C. for 24 hours. Approximately 5×10⁴ DNA strands are attached, as measured by flow cytometry, onto the PS particles, which are 1 μm in diameter. The areal density of DNA strands is 1 strand per nm².

Thermal annealing of the DNA-coated PS particles at 0.5-3° C. below the DNA-hybridization melting temperature results in bulk colloidal crystals in three-dimensions. FIG. 2C shows optical images of crystals formed by particles functionalized with A and B DNA. Confocal fluorescence images suggest a CsCl-type structure. Much larger FCC crystals are shown in FIGS. 8A-D.

PMMA particles. In another embodiment, the colloidal particles are PMMA particles. Following a similar method, i.e. two-stage swelling and co-polymerization, chlorine-coated PMMA particles were synthesized with SDS, MMA and AIBN as the surfactant, monomer and initiator, respectively. 3-chloro-2-hydroxypropyl methacrylate (CHPMA) is used as the co-monomer and chlorine source for two reasons. It is structurally similar to MMA. The amphiphilic nature of this co-monomer facilitates the enrichment of chlorine groups on the particle surface. Indeed, a DNA areal density of 1/27 nm² is achieved. The FCC crystals and CsCl crystal using 500 nm P-PMMA particles or 750 nm A- and B-PMMA particles are shown in FIG. 2D.

Silica particles. In another embodiment, the colloidal particles are silica colloids. Colloidal particles made from silica have Si—OH groups on their surface, which serve as anchor points for further modifications. Silica particles of various sizes were synthesized using the Stöber sol-gel method (FIG. 3A). The resulting particles, which have smooth surfaces, as shown in FIG. 3B, are subsequently treated with (3-bromopropyl)trichlorosilane (BTS). BTS is highly reactive toward Si—OH groups; it coats the silica surfaces with bromine groups with high efficiency (FIG. 3A). The conversion of bromine to azide and the attachment of DNA with sticky ends using click chemistry are conducted as described above. Using this method, 5×10⁴ DNA strands are grafted onto each 1-μm silica particle, which corresponds to an areal density of DNA strands of 1/63 nm². Particles functionalized with ssDNA with complementary sticky ends are mixed, forming a variety of colloidal superlattices including FCC and AB CsCl-type crystals (FIGS. 3D-E).

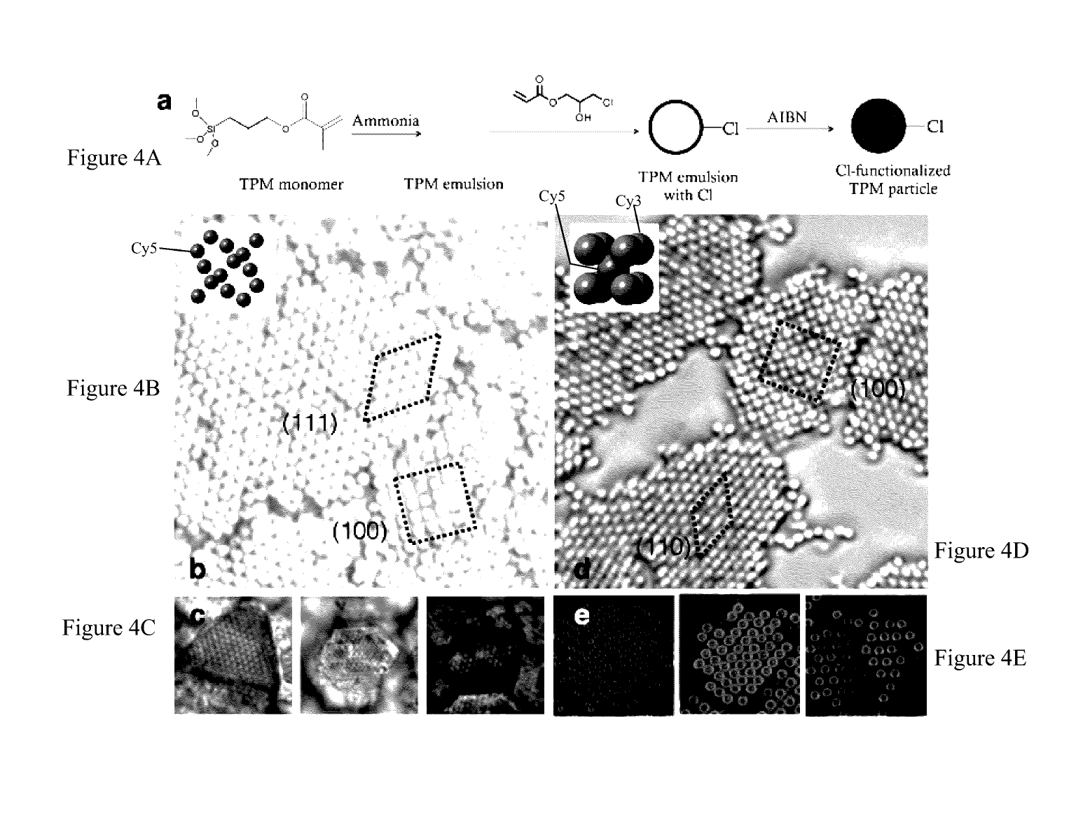

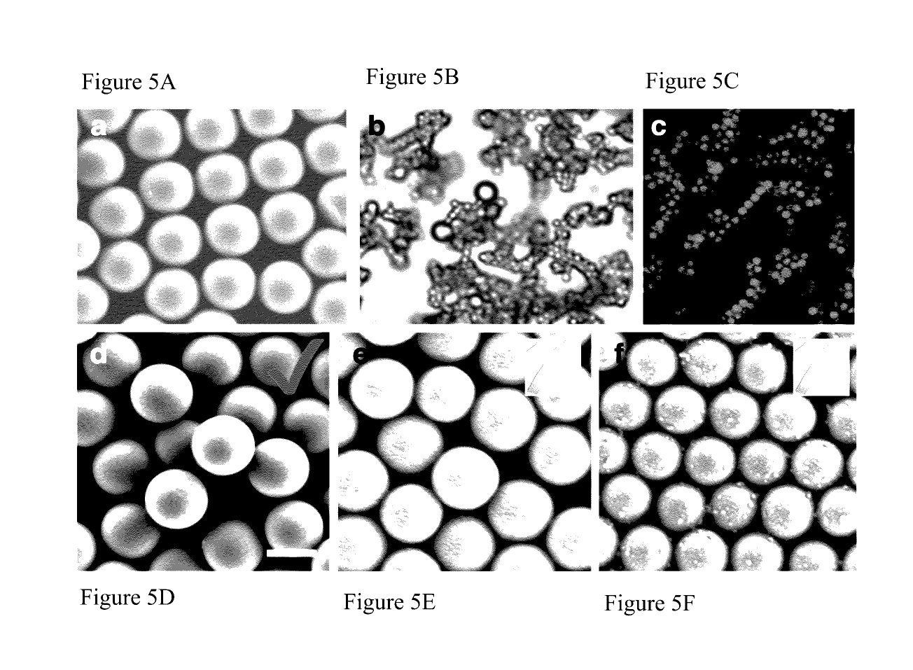

TPM particles. Particles possessing azide anchors for covalent DNA attachment are fabricated by copolymerizing 3-(trimethoxysilyl)propyl methacrylate (TPM) with 3-chloro-2-hydroxypropyl methacrylate (CHPMA) followed by azide substitution of the chlorine groups. Typically, 200 μL of TPM is added to 20 mL of aqueous solution containing ammonium hydroxide (1% w/w). The reaction is allowed to stir for four hours at room temperature, producing monodisperse TPM emulsions. Then, 40 μL of CHPMA is added, which diffuses into the TPM emulsion droplets. For the sphere used in confocal videotaping, 5 mg Coumarin modified styrene monomer or Rhodamine modified methacrylate monomer is added with CHPMA. After 30 minutes, 5 mL of an aqueous solution of sodium dodecyl sulfate (SDS, 5% w/w) is introduced. Ten minutes later, 10 mg of azobis(isobutyronitrile) (AIBN) is added and the reaction mixture is allowed to stir for another 20 minutes before the temperature is raised to 80° C. Thermal degradation of AIBN initiates the polymerization, generating the chlorine-functionalized particles. To make particles with rough surfaces, CHPMA and AIBN are premixed with TPM before hydrolysis and condensation. The emulsion is directly solidified without adding SDS to stabilize the emulsion. The resulting particles are purified by repeated centrifugation/redispersion and finally dispersed in 20 mL of an aqueous solution of Pluronic F127 (0.2% w/w) containing 500 mg of sodium azide (NaN₃) and catalytic amount of potassium iodine (KI). The suspension is then heated at 70° C. for twelve hours, yielding azide functionalized TPM particles. The particles are washed and stored in DI water for further usage. Varying the TPM amount (CHPMA is kept at 20% v/v to TPM), particles of different sizes (d=0.5 μm-3.5 μm) are obtained with low size distribution (<5%). Those particles can readily form bulk crystals. FIGS. 4B-E shows the FCC and CsCl crystals out of DNA-coated TPM particles.

### Colloidal Crystal with Hybrid Material

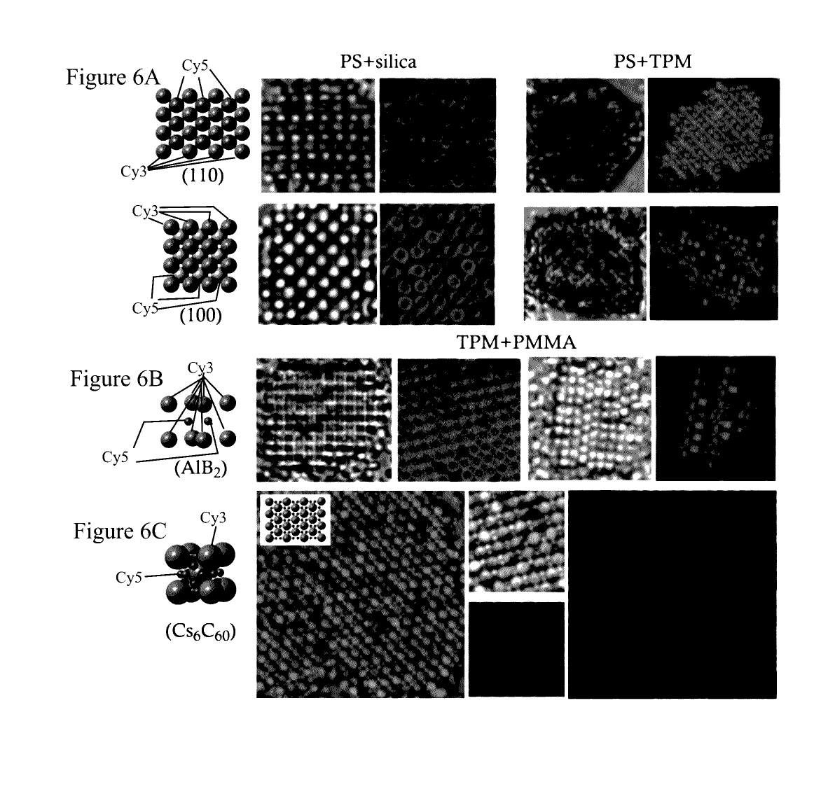

Colloids of any type of materials demonstrated before can in principle be mixed to produce colloidal crystals. For example, one can also fabricate PS-TPM crystals and PS-PMMA crystals, as shown in FIG. 4B and FIGS. 9A-D Using colloids of similar sizes, CsCl-type crystals are formed. It is also demonstrated that the particle compositions and sizes can be altered at the same time resulting in binary crystals isostructural to AlB2 and Cs₆C₆₀. This is shown by combining 500 PMMA particle with 1.0 TPM or 1.5 TPM particles with complementary DNA sticky ends (FIG. 6).

The underlying challenge in materials science is to precisely control the spatial arrangement of the desired composition at nanometer and micrometer scales. DNA-coated colloids are employed whose size, kind and interaction can be programmed at will. Apart from homogenous crystal structures fabricated using particles of a single component, heterogeneous structures can be built. By mixing and annealing PS and silica microspheres coated with complementary DNA, AB CsCl-type crystals are obtained. Note that this crystal is truly binary, with two different materials, organic and inorganic, arranged in an ordered array. Because the refractive index is very different for PS and silica, bright-field optical images of the crystals show sharp contrast between the two components. In FIG. 4A, the 110 and 100 plane of the crystals is shown. In terms of photonic properties, this crystal structure can be treated as a cubic lattice for each of the low and high refractive index materials. On the other hand, silica particles are much heavier than the PS ones, leading to a difference for the number of crystalline planes observed. The 100 planes are dominant at low annealing temperature with the silica particles being the bottom layer, while at higher temperature, 110 planes start to emerge and enrich (FIGS. 8A-C).

### Surface Smoothness

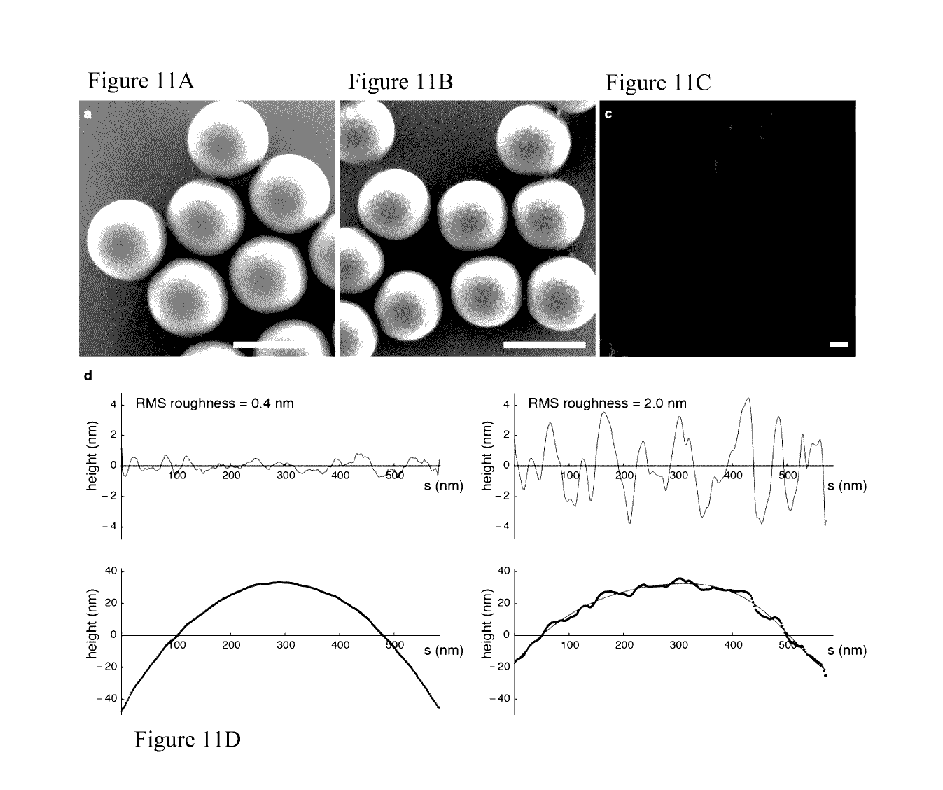

To investigate the effect of surface morphology on crystallization, 1.0-μm TPM particles with rough surfaces were prepared(see FIG. 11B). The root mean square (RMS) fluctuations in surface height for the rough particles are measured with atomic force microscopy (AFM) to be 2.0±0.5 nm, as compared to the smooth particles with RMS height fluctuations of less than 0.5 nm (FIG. 11B). The particles are functionalized with sticky-ended DNA (CGCG), with a DNA surface coverage of 9.1×10⁴ DNA per particle. After repeated annealing near the melting temperature for 24 hours, no crystalline structure is observed, in contrast to particles prepared with smooth surfaces. It is believed that surface roughness creates local free energy minima with large barriers to rolling.

### DNA Areal Density

To investigate the dependence of crystallization on the areal density of ssDNA on the colloids, different sets of 1.0-μm TPM particles are prepared where the particles are fully functionalized with DNA, but only a fraction of the DNA has sticky ends. The remainder of the DNA has the same 61-base poly-T sequence but lacks the sticky ends. Here 8-base palindrome sticky ends were used and it was found that all the samples crystallize after ten hours of annealing when the coverage is equal to or greater than 25%, or about 28,000 DNA strands per particle. When the coverage is 10%, or about 11,500 DNA strands per particle, only about 15% of the particles form crystals; at 5% coverage, particles still aggregate but only about 3% form crystals. These data are summarized in FIG. 9H.

The lateral reach and the areal density of the grafted ssDNA ends determine how many potential partners a sticky end can have on an adjacent colloid. Using literature values for the distance between nucleotides b₀=0.63 nm and the persistence length Lₚ=2.5 nm (for 100 mM NaCl), the distribution of end heights is about 13-17 nm for 61-base ssDNA strands, depending on the exact choice for the excluded volume. The lateral reach Rₗ of the DNA ends should be Gaussian distributed, given by Rₗ≈\sqrt{2LLₚ}=14 nm, where L=Nb₀=38 nm. FIG. 9H plots the mean distance d between active sticky ends and reveals that the particles fail to completely crystallize when d exceeds Rₗ. In this limit, ssDNA strands with sticky ends cease to reach more than one complementary sticky end on the bound particle, which suppress particles from rolling on each other.

Single stranded DNA (ssDNA) consisting of 61 bases is grafted to the surfaces of the colloidal particles. The distance between nucleotides for ssDNA is b₀=0.63 nm which gives a contour length of L=38.4 nm. The persistence length Lₚ=2.5 nm for 100 mM NaCl. In this case the mean squared end-to-end length given by the worm-like chain model is

$${R}^{2} = 2 {LL}_{p} [ 1 - \frac{{L}_{p}}{L} ( 1 - {\text{?}}^{- L / {L}_{p}} ) ] , \\ \text{?} \text{indicates text missing or illegible when filed}$$

which gives R=13 nm. The areal density of DNA is measured by flow cytometry to be σ=1 ssDNA/27 nm², which corresponds to a mean distance of d=5.2 nm between grafting points. Because d<R, the grafted chains are stretched. For stretched chains, Milner, Witten, and Cates give the following expression for the distribution of end heights

$$n ( z ) = \frac{{π}^{2} z \sqrt{{h}^{2} - {z}^{2}}}{2 {w ( L / {L}_{p} )}^{2}} ,$$

where z is the distance from the colloid surface, w is the excluded volume parameter, and

$$h = ( L / {L}_{p} ) {( 12 σ w / {π}^{2} )}^{1 / 2} .$$

Taking the excluded volume to be

$$w \approx {λ}_{p } {L}_{p}^{2} ,$$

where λ_{D} is the Debye length, about 1 nm at 100 mM NaCl, the height h of the ssDNA brush is about 13-17 nm, depending on the precise value of w. Recent AFM measurements of brush heights at comparable areal density of 44-base ssDNA give a height of approximately 8 nm, which extrapolating to 61-base ssDNA gives a height of 11 nm, somewhat smaller but consistent with the above estimate given the uncertainties in the experimental parameters and approximate nature of the models.

The extent of the lateral reach of the DNA ends should be Gaussian distributed with the root mean square distance given by Rₗ≈\sqrt{2LL_{F}}=14 nm, as there is no stretching of the chains in the lateral direction (in contrast to the vertical direction). FIG. 9H plots the mean distance d between active sticky ends and reveals that the particles fail to completely crystallize when d exceeds Rₗ. In this limit, ssDNA strands with sticky ends cease to be able to reach more than one ssDNA sticky end on the particle to which it binds, which would be expected to suppress bound particles from rolling on each other.

### Temperature Study

Because the colloids described above are big enough to be viewed under an optical microscope, it is possible to observe and follow the crystallization process including nucleation, growth, aggregate restructuring, and defect formation of both single component and binary colloids made from different materials. This allows for study of the behavior or the structures during formation. When quenched from above to below the melting temperature, the rate of crystal formation exhibits the familiar maximum for intermediate temperature quenches observed in metallic alloys, but over a temperature range smaller by two orders of magnitude, owing to the highly temperature-sensitive diffusion between aggregated DNA-coated colloids. These results provide new insights into the nucleation and growth of DNA-coated colloids and open the door to programmable colloidal structures.

### DNA Functionalization

Single-stranded oligonucleotides with sticky ends (Integrated DNA Technologies USA) are used in this study. 5′-Amino-DNAs are purchased and the amine groups are converted to a dibenzyl cyclooctane (DBCO) group by treating the DNA with DBCO-sulfo-NHS (Click Chemistry Tool) in phosphate buffered saline (PBS, 10 mM, pH 7.4, 100 mM salt, same below). The DNA is also internally fluorescent labeled with Cy3 (emission maximum 564) or Cy5 (emission maximum 668), respectively. Both palindrome (P) and complementary (A/B) DNA are used, with the length of sticky end containing four or eight bases. The sequences are:

A4:
5′-/DBCO/(T)₂₀-Cy3-(T)₄₁-GCAG-3′
(SEQ ID NOS 1 and 2, respectively, in order of
appearance)
B4:
5′-/DBCO/(T)₂₀-Cy5-(T)₄₁-CTGC-3′
(SEQ ID NOS 1 and 3, respectively, in order of
appearance)
P4:
5′-/DBCO/(T)₂₀-Cy5-(T)₄₁-CGCG-3′
(SEQ ID NOS 1 and 4, respectively, in order of
appearance)
P8:
5′-/DBCO/(T)₂₀-Cy5-(T)₄₁-CGTATACG-3′
(SEQ ID NOS 1 and 5, respectively, in order of
appearance)

In a typical DNA grafting experiment, azide functionalized particles are first dispersed in 400 μL of PBS containing Triton X-100 (0.1% w/w) with a particle concentration of 0.1% w/w. Then, 20 μL of DBCO-DNA (100 μM) is added to the particle suspension and the reaction mixture is stirred at 55° C. for 24 hours, yielding the DNA-functionalized particles. The particles are washed and stored in PBS containing 1% w/w Pluronic F127 for the self-assembly experiments.

### Flow Cytometry

Flow cytometry was used to quantify the number of DNA strands functionalized per particle. Cy5-labeled microsphere are used as cytometry standard (Quantum™ Cy™5 MESF, Bangs Laboratories Inc.). Using the provided molecules of equivalent soluble fluorochromes (MESF), a calibration curve is constructed, based on which the measured fluorescent intensity data for each of DNA-coated particle sample is converted to an approximate number of DNA grafted on each particle. Flow cytometry experiments are carried out using a BD LSRII HTS cytometer. Particle samples are dispersed in PBS with Pluronic F127 (1% w/w).

### Diffusion Experiment

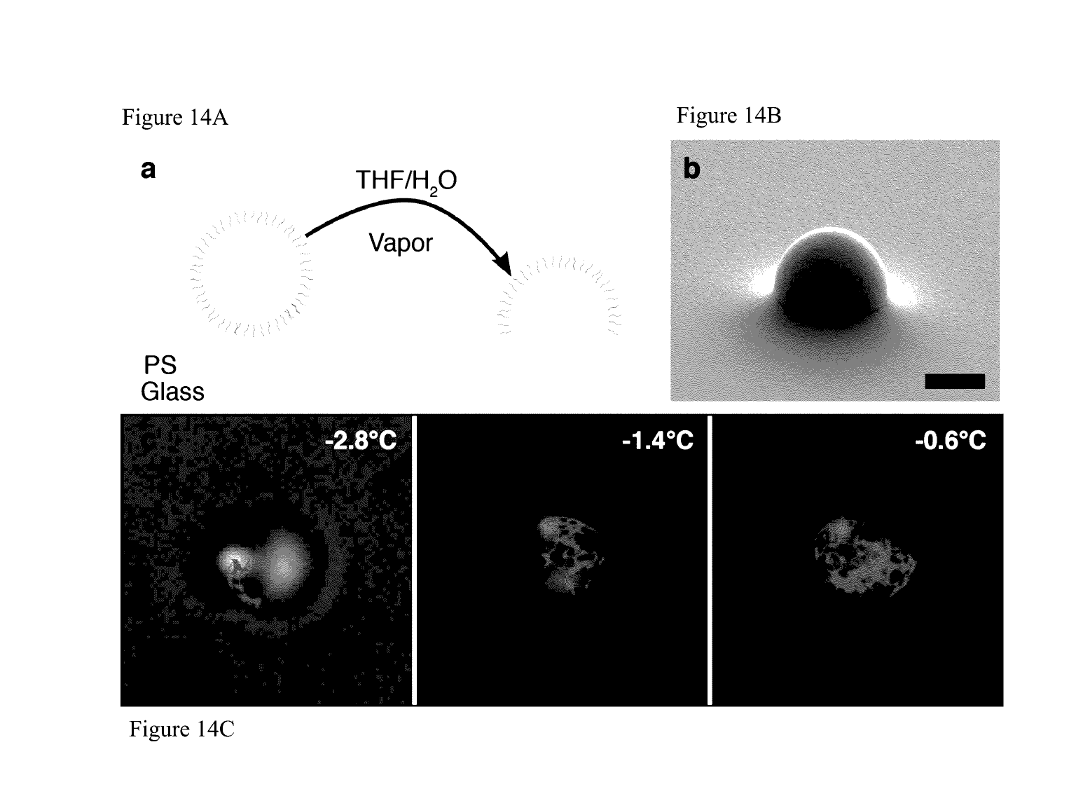

To investigate the mobility of bound DNA-coated colloids, 2.0-μm DNA-coated colloidal particles were affixed to a glass microscope slide by embedding them in a thin polystyrene film spin coated on the slide (FIG. 14). The slide forms the one side of a cell, which is subsequently filled with a very dilute suspension of 1.0-μm colloidal particles coated with DNA strands complementary to those on the immobilized 2.0-μm particles. The sample is heated above the melting temperature and then cooled below the melting temperature. After a period of time, a free 1.0-μm DNA-coated colloidal particle binds to an immobilized 2.0-μm particle attached to the substrate. The motion of the 1.0-μm particle is tracked as it diffuses on the sphere for 6 minutes at 5 frame/s.

### Measurements of Particle Roughness

The roughness of the particle surfaces was measured with a tapping mode atomic force microscopy (AFM) in air. Results of typical scans of a smooth (left plots) and a rough particle (right plots) are shown in Extended Data FIG. 11D.

### Self-Assembly

For the self-assembly studies, the particles of interest were combined, mixed according to the stoichiometry of the target crystalline structure, and transferred to a glass capillary tube (2 mm×100 μm×10 cm). The capillary tube was pretreated with oxygen plasma for one minute and exposed to hexamethyldisilazane vapor to render it hydrophobic to prevent DNA-coated colloids from sticking. After adding the sample, the capillary tube is sealed and attached to a microscope glass slide using wax. The slide is then mounted on a homemade microscope thermal stage with the ability to create a temperature gradient. For crystal growth, the sample is first heated above the melting temperature to melt any aggregates and then quenched to different temperatures below Tₘ and held constant.

Crystals are observed to form only when the number ratio of particles mixed is near the stoichiometry of the target crystalline structure. For example, when making Cs₆C₆₀ crystals, the number ratio of 0.54-μm particles to 1.5-μm particles is kept around 6:1. Slight changes in this ratio still result in the same crystal structure but with a different amount of crystal vacancy defects. For instance, when the ratio is 4:1 (insufficient 0.54-μm particles), there are many vacancies for the smaller particles. When the ratio is increased to 8:1, very few vacancies are found. For greater degrees of non-stoichiometric preparations, crystals are not observed.

### Microscopy

Bright-field optical images and videos were obtained using a Nikon TE300 microscope equipped with a CCD camera. Fluorescent images and videos were taken using a Leica SP8 confocal fluorescence microscope. Some of the microscope images and videos were digitally post-processed to improve brightness and contrast.

### Mobility of Bound DNA-Coated Colloids

The ability to roll and rearrange is critical for maximizing the DNA hybridization between bound particles and for the formation of crystalline structures. To investigate the mobility of bound DNA-coated colloids, the motion of a 1.0-μm B particle is tracked as it diffuses on an immobilized 2.0-μm A particle (FIG. 14). The mean square displacement [symbol] r² [symbol] of a particle on a sphere is well described by [symbol] r² [symbol] =At^{α} where 0.7<α≦1, as shown in FIG. 3e. For temperatures just below Tₘ, α is very nearly 1, which corresponds to normal diffusive motion. For lower temperatures, α<1, which means the particle motion is subdiffusive. This can occur when there is a random distribution of traps, which are expected from random fluctuations in DNA grafting density on the colloids.

After binding to each other, particles need not diffuse far to crystallize. Assuming the typical distance to be of the order of a particle radius R, the characteristic time τ can be read off the horizontal gray line at [symbol] r² [symbol] =R²=0.28 μm² in FIG. 3e for each data set. This time increases rapidly with quench depth: τ=2.5 s, 11 s, and 46 s for quench depths of −0.6° C., −1.7° C., and −2.8° C., respectively. This decrease in particle mobility is due in part to the fact that particles diffuse more slowly when their binding energy increases as the temperature is lowered. The decrease is exacerbated by the fact that the particle motion becomes increasingly subdiffusive—α decreases—as the temperature is lowered.

### Crystallization Kinetics

Crystal formation kinetics depend sensitively on the quench depth. FIGS. 9A-D show the formation of CsCl crystals at different annealing temperatures. At an intermediate temperature (27.5° C.), it takes only 45 minutes to crystallize most of the particles (>90%). At higher temperatures (28.3° C. and 27.9° C.) and at a lower temperature (27.0° C.), the time required to reach 90% conversion is significantly longer. These results are summarized in FIG. 9F which plots the annealing temperature vs. the time required to attain 5%, 50%, and 90% crystal conversion—the time-temperature-transformation (TTT) diagram—for the CsCl structure. The curves display the same characteristic “C” shape observed in metallic alloys, where the nucleation and overall transformation rate exhibit a maximum for an intermediate temperature quench.

For the shallowest quenches, crystal formation proceeds by nucleation and growth. A time-lapse video at an annealing temperature of 28.3° C. (T-Tₘ=−0.6° C.) revealed incipient clusters of up to 6-8 particles across that form and fade over a period of minutes, indicating that the system is metastable. Eventually, a larger crystalline cluster appears after about 45 minutes (00:45:00), some 15 particles across, and grows. As it grows, other incipient clusters appear and fade away. Eventually, some five large separate crystals nucleate and grow to encompass nearly all the available particles (03:00:00) in the field of view.

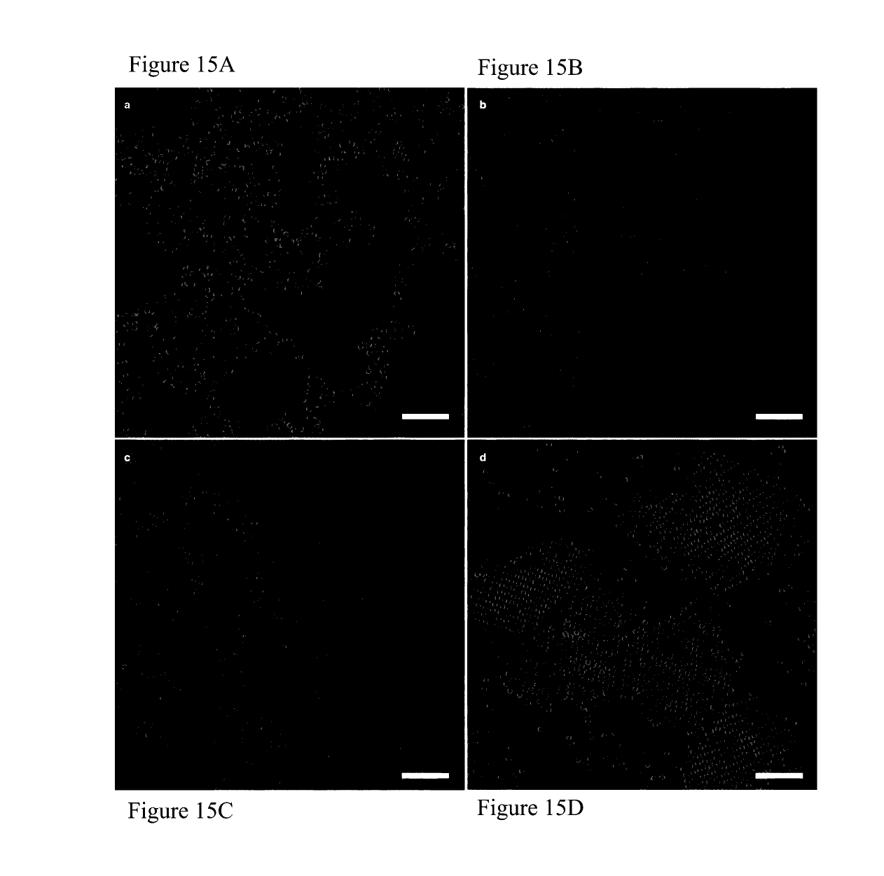

At 27.9° C., crystal formation proceeds by a similar process, although the overall transformation time is significantly shorter than for the shallower quench to 28.3° C., due to a much faster nucleation rate. This is consistent with classical nucleation theory, in which both the free energy barrier and the critical nucleus size decrease as the quench depth is increased. Once nuclei form, the crystals actually grow more slowly than they do for the shallower quench, as revealed by the data in FIG. 9G. This is attributed to the slower rate at which particles diffuse and roll to find their lattice positions after binding to a crystal. Because crystals nucleate so much faster, many more nucleate, which makes their ultimate sizes much smaller than observed for the shallower quench (FIG. 15).

For the deepest quenches, crystal formation proceeds by a two-stage process in which a dense amorphous aggregate forms very rapidly, followed by slow crystallization. When the system is quenched to 27.0° C. (T-Tₘ=−1.9° C.), density fluctuations appear almost immediately on length scales much larger than the particle size, which suggests that the system is globally unstable and undergoes spinodal decomposition. Very soon thereafter, a dense metastable amorphous network forms. Subsequently, particles in the network rearrange locally as small crystals form and grow throughout the sample, which results in a large polycrystalline aggregate consisting of approximately a hundred crystallites in the field of view. Here crystals grow by local rearrangements that occur by diffusion, which is very slow for these deep quenches. So even though the first crystals appear very early after the quench, as indicated by the 5% conversion time in the TTT diagram in FIG. 9F, the system proceeds to 90% conversion much more slowly than it does for shallower quenches. This is also reflected in the very slow crystal growth rate, 0.8 particle/min shown in FIG. 9F for the deepest quench. At 27.5° C., phase separation also proceeds by the two-stage process of spinodal decomposition to an amorphous aggregate followed by crystallization via local rearrangements. However, particle diffusion within the amorphous aggregate is much faster than it is for the deepest quench discussed above. Thus, the overall time for crystallization is shortest for this intermediate quench.

The time for the initial aggregates to form, whether they are ordered or amorphous, decreases with increasing quench depth (Table 1). On the other hand, the time it takes for crystals to grow gets steadily longer as diffusion slows with increasing quench depth. The net result is that the overall crystal formation occurs fastest for intermediate quenches (FIG. 3f). This is similar to what is observed in metallic alloys, where the crystallization rates change over temperature scales of several hundred degrees. Here, by contrast, the changes occur over a few degrees.

TABLE 1
Aggregation time of DNA coated colloids at various
temperatures. The time required for DNA-coated colloids
to form stable or metastable clusters after a rapid quench
from above the melting temperature Tₘ = 28.7° C.

| Temperature (° C.) | 27 | 27.5 | 27.9 | 28.3 |
|---|---|---|---|---|
| Aggregation time (min) | 3.1 ± 0.2 | 3.7 ± 0.2 | 14 ± 1 | 45 ± 2 |

The crystal formation process for the FCC, AlB₂, and Cs₆C₆₀ exhibits a similar temperature dependence trend but proceeds at different rates. Palindrome particles crystallize fastest, forming an FCC lattice. For example, 0.54-μm P Particles nucleate within five minutes, and 90% conversion is achieved in 15 minutes at an intermediate quench temperature. The nucleation rate of AlB₂ and Cs₆C₆₀ is slowed as each particle can bind only with a fraction of the population. Increasing particle size also slows crystallization as larger particles have slower diffusion rates. Increasing the number of bases in the DNA sticky ends slows the crystallization kinetics. For example, changing the length of the sticky ends of DNA on 0.54-μm particles from four (CGCG) to eight (CGTATACG) bases, both palindrome sequences, increases the time required for 90% conversion at intermediate temperature quenches by a factor of four.

### Crystal Defects

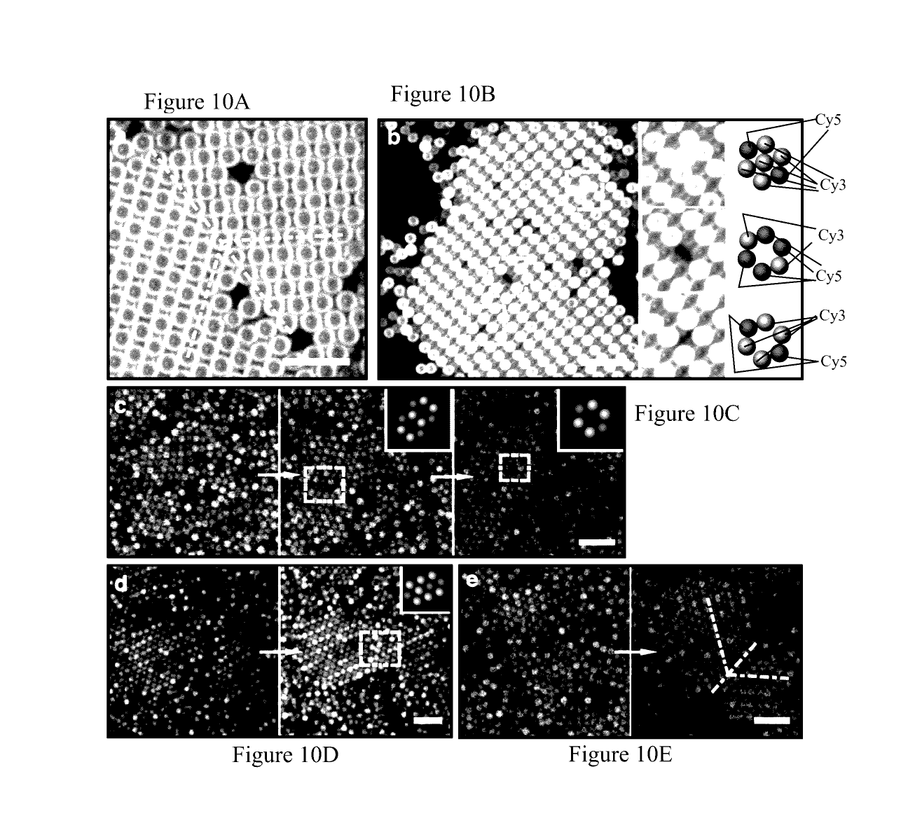

Different kinds of defects in were observed in the crystals, including vacancy defects, antisite defects, and grain boundaries. FIG. 10A shows a twin boundary on the 110 plane of a Cs₆C₆₀ crystal while FIG. 10B shows a variety of vacancy defects and an antisite defect. To observe the formation of these various defects, the crystallization of fluorescent TPM particles is followed using confocal microscopy. It was found that single particle vacancy defects form as the crystals grow while larger vacancy defects usually form when two crystals merge with their crystalline axes aligned (FIG. 10C). Antisite defects form when a particle is trapped in the “wrong” site during crystal merging or rapid crystal growth. In FIG. 10D, a case is shown where a green particle present at the junction between two merging crystals is trapped in the wrong position, finding no suitable adhesion point. Grain boundaries develop when two crystals approach each other with misaligned crystalline axes, and are often accompanied by vacancy defects. FIG. 10E shows the formation of grain boundaries in a CsCl lattice.

The ability of DNA-bound particles to diffuse and anneal means that DNA-coated colloids can surmount kinetic barriers and find pathways to form the structures they have been programmed to create. This opens up the study of self-assembly and defect formation in situ using conventional optical microscopy, providing an attractive model platform to study the self-assembly of particulate systems.

Moreover, using colloidal building blocks where the DNA coating is much thinner than the particle size makes possible a new materials science in which particles, and not DNA, constitute the majority component of the structure. Thus DNA becomes a structure-directing glue for putting together different materials. Extending these techniques to make not only the binary crystals illustrated here, but to make much more complex structures out of different materials—plastics, inorganics, metals, and semiconductors—is well within reach. As noted above, different materials may be used, such as polystyrene, poly(methylmethacrylate), and silica colloids with smooth surfaces and azide functional groups. Further, structure may be made using a mixture of different colloidal particles. High areal density ssDNA can be similarly grafted onto these particles using SPAAC; single component and heterogeneous binary colloidal crystals have been made from these materials.

Because these techniques can achieve areal densities of ssDNA exceeding 10⁵ ssDNAs per micrometer-sized particle, far in excess of the 10⁴ ssDNA required for particles to diffuse and anneal, different particles can be coated with many different ssDNA sticky-end codes, which should facilitate the programmed assembly of structures much more complex than the binary crystals demonstrated here. The ability for DNA-bound particles to anneal should also facilitate assembling patchy colloidal particles into more open complex colloidal architectures.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for fabricating coated colloidal particles comprising:
synthesizing a plurality of micrometer size colloidal particles; and
coating the plurality of colloidal particles with nucleic acids by applying strain-promoted alkyne-azide cycloaddition;
wherein the particle comprises dense and homogenous chlorine/azide surface functionalities.

2. The method of claim 1, wherein the colloidal particles comprise materials selected from the group consisting of poly(styrene), poly(methylmethacrylate) (PMMA), silica and titania, and 3-(trimethoxysilyl)propyl methacrylate (TPM).

3. The method of claim 1, wherein the particles comprise particles of different sizes with smooth surface morphology and a size distribution of <5%.

4. The method of claim 1, wherein the particles have a size range of diameter=0.5 μm-3.5 μm.

5. The method of claim 1, further comprising providing a salt in a solution with the colloidal particles prior to coating with nucleic acids.

6. The method of claim 5, wherein providing the salt comprises providing 100 mM solution of sodium chloride.

7. The method of claim 5 further comprising adding a surfactant to the solution prior to coating.

8. The method of claim 1, wherein coating comprises greater than 10⁵ nucleic acid strands per particle.

9. A colloidal crystal composition comprising:
a colloidal particle having a plurality of strands of DNA attached thereto;
each strand of DNA comprising a 5′ end attached the colloidal particle, a 3′ terminus comprising a sticky end; and a flexible spacer extending therebetween comprising a plurality of base pairs;
wherein a plurality of colloidal particles are bound by binding of respectively associated DNA strands at the 3′ sticky end.

10. The composition of claim 9, wherein the colloidal particle is polystyrene.

11. The composition of claim 10, wherein areal density of the plurality of DNA strands is at least 1 strand per 63 nm².

12. The composition of claim 9, wherein the colloidal particle is PMMA.

13. The composition of claim 10, wherein areal density of the plurality of DNA strands is at least 1 strand per 27 nm².

14. The composition of claim 9, wherein the colloidal particle is silica.

15. The composition of claim 10, wherein areal density of the plurality of DNA strands is at least 1 strand per 63 nm².

16. The composition of claim 9, wherein the colloidal particle is TPM.

17. The composition of claim 10, wherein areal density of the plurality of DNA strands is at least 1 strand per 27 nm².

18. A method to crystallize micrometer-sized colloidal particles driven by nucleic acid interaction; comprising
forming a first plurality of colloidal particles having a first size;
attaching a plurality of a first single-stranded nucleic acid to each of the first plurality of colloidal particles;
forming a second plurality of colloidal particles having a second size;
attaching a plurality of a second single-stranded nucleic acid to each of the second plurality of colloidal particles, the first single-stranded nucleic acid and the second single-stranded nucleic acid being complementary;
thermally annealing the first single-stranded nucleic acid and the second single stranded nucleic acid at a temperature below hybridization temperature.

19. The method of claim 18, wherein thermally annealing comprises quenching temperature to 0.5-3° C. below the hybridization temperature.

20. The method of claim 19, wherein the first plurality of particles and the second plurality of particles have root mean square fluctuations in surface height of less than 0.5 nm.


SEQUENCE LISTING (9 sequences)

SEQ ID NO: 1 - DNA, 20 bp, Artificial Sequence
  Description of Artificial Sequence Synthetic oligonucleotide
tttttttttttttttttttt

SEQ ID NO: 2 - DNA, 45 bp, Artificial Sequence
  Description of Artificial Sequence Synthetic oligonucleotide
tttttttttttttttttttttttttttttttttttttttttgcag

SEQ ID NO: 3 - DNA, 45 bp, Artificial Sequence
  Description of Artificial Sequence Synthetic oligonucleotide
tttttttttttttttttttttttttttttttttttttttttctgc

SEQ ID NO: 4 - DNA, 45 bp, Artificial Sequence
  Description of Artificial Sequence Synthetic oligonucleotide
tttttttttttttttttttttttttttttttttttttttttcgcg

SEQ ID NO: 5 - DNA, 49 bp, Artificial Sequence
  Description of Artificial Sequence Synthetic oligonucleotide
tttttttttttttttttttttttttttttttttttttttttcgtatacg

SEQ ID NO: 6 - DNA, 54 bp, Artificial Sequence
  Description of Artificial Sequence Synthetic oligonucleotide
ttttttttttttttttttttttttttttttttttttttttttttttttttcgcg

SEQ ID NO: 7 - DNA, 34 bp, Artificial Sequence
  Description of Artificial Sequence Synthetic oligonucleotide
ttttttttttttttttttttttttttttttgcag

SEQ ID NO: 8 - DNA, 34 bp, Artificial Sequence
  Description of Artificial Sequence Synthetic oligonucleotide
ttttttttttttttttttttttttttttttctgc

SEQ ID NO: 9 - DNA, 41 bp, Artificial Sequence
  Description of Artificial Sequence Synthetic oligonucleotide
ttttttttttttttttttttttttttttttttttttttttt